(19) United States
(12) Patent Application Publication
(10) Pub. No.: US 2003/0105471 A1
(43) Pub. Date: Jun. 5, 2003

(54) Plug-type connection for releasably connecting two bodies

(76) Inventors: Fridolin Schlapfer, Glarus (CH); Martin Hess, Holstein (CH)

Correspondence Address: PENNIE & EDMONDS LLP, 1667 K STREET NW, SUITE 1000, WASHINGTON, DC, 20006

(21) Appl. No.: 10/291,386

(22) Filed: Nov. 12, 2002

Related U.S. Application Data
(63) Continuation of application No. PCT/CH00/00263, filed on May 11, 2000.

(51) Int. Cl. A61B 17/58

(52) U.S. Cl. 606/104; 606/73

(57) ABSTRACT

A plug-type connection for releasably connecting two bodies (1) and (2),

A) the first body (1) comprising M (M≧1) first connection segments (5.i) arranged coaxially to a central axis (4);

B) the second body (2) comprising N (N≧1) second connection segments (7.j) arranged coaxially to said central axis (4), so that

C) the first connection zone (3) and the second connection zone (6) form a plug-type connection extending coaxially to said central axis (4),

D) the first connection zone (3) having at least one first connection segment (5.1) with a non-circular cross-section Q_{5.1} and the second connection zone (6) having at least one second connection segment (7.1) with a cross-section Q_{7.1} in positive engagement with said cross-section Q_{5.1}, whereby a rotationally stabilised, positive engagement relative to the central axis (4) is achievable;

E) M×N being ≧2; and

F) at least one first connection segment (5.k) and at least one second connection segment (7.1) forming an axially non-positive engagement relative to the central axis (4).

The invention relates to a plug-type connection for releasably connecting two bodies as claimed in the precharacterising part of claim 1.

For a minimally invasive and endoscopic use of surgical instruments and implants it is extremely important to establish a firm connection between screw and screwdriver. With screws having large head dimensions firm connections are no problem.

A connection of this type between a pedicle screw or a spinal column retractor and a manipulation device is disclosed in WO 94/26190 SCHLÄPFER. This known connection, according to one of its embodiments, comprises a connecting mechanism which includes a bore open towards the top-side and having an internal screw thread and a slot formed in the head of the pedicle screw and extending transversely to the axis of the bore, and an external screw thread formed in the manipulation device and corresponding to said internal screw thread as well as two cams corresponding to said slot. In this way, it is possible to obtain a firm connection with respect to rotation and axial displacement. Further embodiments include a bayonet locking, a dovetail-like joint which requires a lateral insertion of the manipulation device into the screw head and which permits, once the manipulation device is inserted, a releasable locking by means of a pin extending concentrically to the axis of the screw and displaceable away from and back to the manipulation device, and a connection including two cams formed in the front end portion of the manipulation device which engage with a groove formed in the end face of the screw head and presenting a cylindrical relief. A T-shaped slide lodged concentrically to the screw axis in the manipulation device is axially insertable through the groove and, on reaching the cylindrical relief, may be turned about the screw axis so as to form a connection between manipulation device and screw head that is secured against axial displacement.

With small-headed screws having hexagon sockets, problems will arise resulting from the small dimensions. It is, however, possible to use a screwdriver with a conical hexagon insert bit. Another possibility consists in turning the hexagon insert bit of the screwdriver to such an extent that it is still possible, by using some degree of force, to insert the screwdriver into the hexagon socket. The main problems with these solutions consist in a reduced strength of the drive segment and in the risk that while being manipulated the screw may fall off the screwdriver and disappear in the depth of the wound.

The problem of the reduced driving force and the risk of loosing the screw in the wound may be avoided by means of an additional screw holding sleeve. This solution, however, necessitates the use of a second instrument, which makes the manipulation more difficult and causes additional expenditure for instruments. In addition, the screwdriver equipped with a screw holding sleeve requires more space, which entails a higher degree of soft-tissue damage.

The invention is intended to provide a remedy for this. It is accordingly an object of the invention to provide a connection between screw and screwdriver which is resistant to tensile strain, to impact, and the strength of which is not reduced as it is rotated. This is achieved in that the operating end of the screwdriver, which is inserted into the screw head, includes, in addition to the screwdriver's drive segment properly speaking, a segment that may be wedged within the screw head.

According to the invention, this object is achieved by means of a plug-type connection for releasably connecting two bodies which shows the features of claim 1.

The plug-type connection for releasably connecting two bodies according to the invention comprises on one of said bodies a first connection zone including a central axis and M connection segments extending coaxially to the central axis as well as a second connection zone, formed in the second body, including a central axis and N connection segments. The M or respectively N connection segments are coaxially arranged relative to their respective central axes and may be realised on any of the connection zones in the form of plugs and/or recesses, so that the first connection zone and the second connection zone form a plug-type connection extending coaxially to the central axis. In addition, the first connection zone comprises at least one connection segment having a non-circular cross-section and the second connection zone comprises at least one connection segment having a cross-section that is in positive engagement with said non-circular cross-section. Thus it is possible to achieve a rotationally stabilised, positive engagement relative to the central axis. In addition, the plug-type connection provides a non-positive engagement which is effective coaxially to the central axis. Furthermore, concerning the number of connection segments, the following conditions are applicable:

M≧1

N≧1

M×N≧2

The first connection segments are arranged coaxially and differ from each other in shape in the different embodiments of the device according to the invention. These differences in shape may derive from different cross-sections, such as hexagon, star-shaped form with six branches, or area of a circle, or else from different diameters with identical cross-sectional shape. The same is true of the second connection segments of the second connection zone formed in the second body.

In addition to the connection segments permitting a rotationally positive engagement of the two bodies, the plug-type connection according to the invention as realised in further embodiments comprises connection segments which cooperate in such a way as to cause an axially non-positive engagement, and, in yet another embodiment, also permit a stabilisation of the central axis of the plug-type connection, which is of particular interest in the case of a non-positive engagement that is exposed to force. As to the arrangement of the connection segments, the following variants are possible:

| | Connection zone 1 | | | Connection zone 2 | | |
|---|---|---|---|---|---|---|
| | M × N = 2 | | | | | |
| | Connection segments: M = 1 | | | Connection segments: N = 2 | | |
| | Segment with | Segment with | | Segment with | Segment with | |
| | positive | non-positive | Stabilisation | positive | non-positive | Stabilisation |
| | engagement | engagement | segment | engagement | engagement | segment |
| Function: | | | | | | |
| Positive | X | | | X | | |
| engagement | | | | | | |
| Non-positive | X | | | (X) | X | |
| engagement | | | | | | |
| Stabilisation | X | | | X | | |
| | Connection zone 1 | | | Connection zone 2 | | |
| | M × N = 2 | | | | | |
| | Connection segments: M = 2 | | | Connection segments: N = 1 | | |
| | Segment with | Segment with | | Segment with | Segment with | |
| | positive | non-positive | Stabilisation | positive | non-positive | Stabilisation |
| | engagement | engagement | segment | engagement | engagement | segment |
| Function: | | | | | | |
| Positive | X | | | X | | |
| engagement | | | | | | |
| Non-positive | (X) | X | | X | | |
| engagement | | | | | | |
| Stabilisation | X | | | X | | |
| | Connection zone 1 | | | Connection zone 2 | | |
| | M × N = 4 | | | | | |
| | Connection segments: M = 2 | | | Connection segments: N = 2 | | |
| | Segment with | Segment with | | Segment with | Segment with | |
| | positive | non-positive | Stabilisation | positive | non-positive | Stabilisation |
| | engagement | engagement | segment | engagement | engagement | segment |
| Function: | | | | | | |
| Positive | X | | | X | | |
| engagement | | | | | | |
| Non-positive | (X) | X | | (X) | X | |
| engagement | | | | | | |
| Stabilisation | X | | | X | | |
| | Connection zone 1 | | | Connection zone 2 | | |
| | M × N = 6 | | | | | |
| | Connection segments: M = 2 | | | Connection segments: N = 3 | | |
| | Segment with | Segment with | | Segment with | Segment with | |
| | positive | non-positive | Stabilisation | positive | non-positive | Stabilisation |
| | engagement | engagement | segment | engagement | engagement | segment |
| Function: | | | | | | |
| Positive | X | | | X | | |
| engagement | | | | | | |
| Non-positive | (X) | X | | (X) | X | (X) |
| engagement | | | | | | |
| Stabilisation | X | | | X | | X |
| | Connection zone 1 | | | Connection zone 2 | | |
| | M × N = 6 | | | | | |
| | Connection segments: M = 3 | | | Connection segments: N = 2 | | |
| | Segment with | Segment with | | Segment with | Segment with | |
| | positive | non-positive | Stabilisation | positive | non-positive | Stabilisation |
| | engagement | engagement | segment | engagement | engagement | segment |
| Function: | | | | | | |
| Positive | X | | | X | | |
| engagement | | | | | | |
| Non-positive | (X) | X | (X) | (X) | X | |
| engagement | | | | | | |
| Stabilisation | X | | X | X | | |
| | Connection zone 1 | | | Connection zone 2 | | |
| | M × N = 9 | | | | | |
| | Connection segments: M = 3 | | | Connection segments: N = 3 | | |
| | Segment with | Segment with | | Segment with | Segment with | |
| | positive | non-positive | Stabilisation | positive | non-positive | Stabilisation |
| | engagement | engagement | segment | engagement | engagement | segment |
| Function: | | | | | | |
| Positive | X | | | X | | |
| engagement | | | | | | |
| Non-positive | (X) | X | (X) | (X) | X | (X) |
| engagement | | | | | | |
| Stabilisation | X | | X | X | | X |

Variants with M×N=3 are equally possible.

The inventive device presenting one of the plug-type connections comprises as a first body a manipulation device, particularly a screwdriver, including the first connection zone, and as a second body a screw or an osteosynthetic implant, particularly a bone screw, including the second connection zone.

In another embodiment of the device according to the invention the first and the second connection zones are interchanged, so that the manipulation device, in particular the screwdriver, comprises the second connection zone and, vice versa, the screw comprises the first connection zone.

The screwdriver is provided, beside the screwdriver drive segment (e.g. hexagon insert bit) representing a first connection segment, with a frusto-conical segment (screwdriver clamping segment) representing a second connection segment. Complementarily, the drive segment of the screw (e.g. hexagon socket) representing the first connection segment, is additionally provided with a screw clamping segment representing the second connection segment. For reasons of production technology, this screw clamping segment is preferably shaped in the form of a cylindrical bore having a diameter smaller than the maximum diameter of the frusto-conical screwdriver clamping segment and larger than the respective minimum diameter. The angle of the truncated cone is selected sufficiently small (2 to 10 degrees) so that on pressing the frusto-conical clamping segment of the screwdriver into the cylindrical clamping segment of the screw it becomes wedged therein and is retained by friction. This process of becoming wedged has created a connection between screwdriver and screw which is characterised by a good tensile strength without impairing the force of the rotative drive segment.

Wedge-shaped connections suffer from the disadvantage that the connection may abruptly come loose when exposed to bending stress. In the present case, however, the risk of the wedge-type connection coming loose under bending stress is reduced by the action of the rotative drive segment: the greater the length of the drive segment is, the greater will be the stabilising effect.

For reasons of production technology, the drive segment of the screwdriver will always fit in the drive segment of the screw with a certain play and the stabilising effect on the wedge-type connection becomes smaller as the play becomes greater. The negative influence of the play may be eliminated by completing the drive segment of the screwdriver with a cylindrical stabilisation segment representing a third connection zone and by modifying the screw head correspondingly. From the point of view of production technology, the two cylindrical, mating stabilisation segments may readily be provided with a tight fit.

Another possibility of reducing the negative influence of the play in the area of the drive segment consists in adding to the frusto-conical screwdriver clamping segment a cylindrical stabilisation segment having a diameter smaller than or equal to the minimum diameter of said frusto-conical screwdriver clamping segment. In a complementary way, the cylindrical screw clamping segment must accordingly be lengthened by adding a cylindrical screw stabilisation segment. From the point of view of production technology, the two cylindrical, mating stabilisation segments may present a tight fit.

In a further embodiment, the screwdriver comprises an axially resilient pressure pin which extends coaxially to its longitudinal axis. After the screw has been inserted, the pressure pin is pushed forward relative to the screwdriver and pressed against the screw. Thus, the screwdriver may be entirely pressed out of the screw head. It is possible, by means of the pressure pin, to overcome even a strong compressive force generated by the automatic locking of the wedge-type connection and thus to release the connection between screwdriver and screw. In addition, the ejection mechanism presents the following advantage as compared to standard screwdrivers:

In cases in which the posterior column is operated on using an anterior approach, there is a need for very long screwdrivers. Due to the great length of the screwdriver, even a slight pressure exerted by the surrounding soft tissues may cause problems during the removal of the screwdriver, arising from the screwdriver getting wedged in the drive segment of the screw. Using the ejection mechanism, the screwdriver may be removed without any great effort even when exposed to lateral pressure exerted by the surrounding soft tissues.

Further advantageous embodiments of the invention will be characterised in the dependent claims.

The advantages achieved by the present invention consist essentially in the fact that the plug-type connection according to the invention makes it possible:

to establish a shock-resistant connection between screwdriver and screw;

to avoid an impairment of the driving force;

to require only a minimum space (no screw holding sleeve necessary); and

to resolve the problem, due to the ejection mechanism, of the screwdriver getting wedged in the drive segment of the screw.

In the following, the invention and improvements of the invention will be illustrated in greater detail with reference to the partially diagrammatic representations of several embodiments.

In the drawings:

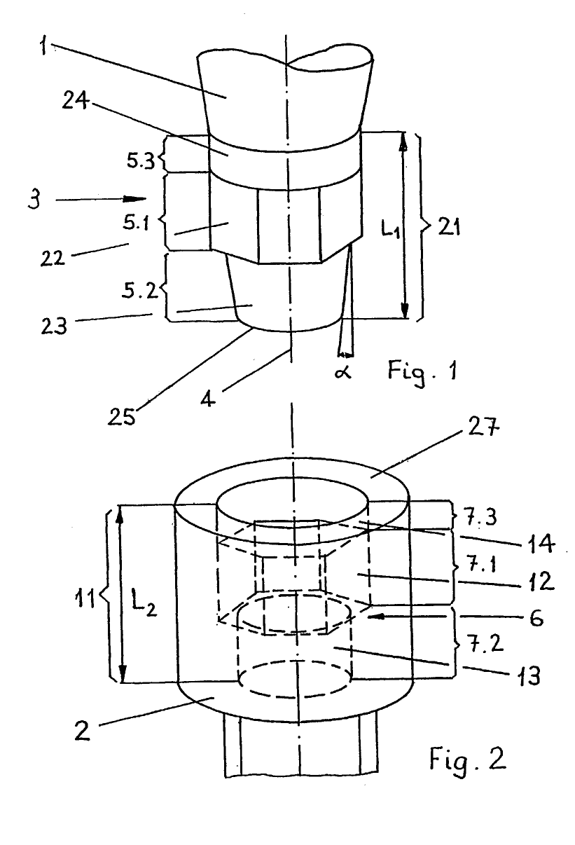

FIG. 1 shows a perspective view of a tenon-like connection zone according to one embodiment of the inventive, plug-type connection;

FIG. 2 shows a perspective view of a connection zone shaped in the form of a recess according to one embodiment of the inventive, plug-type connection;

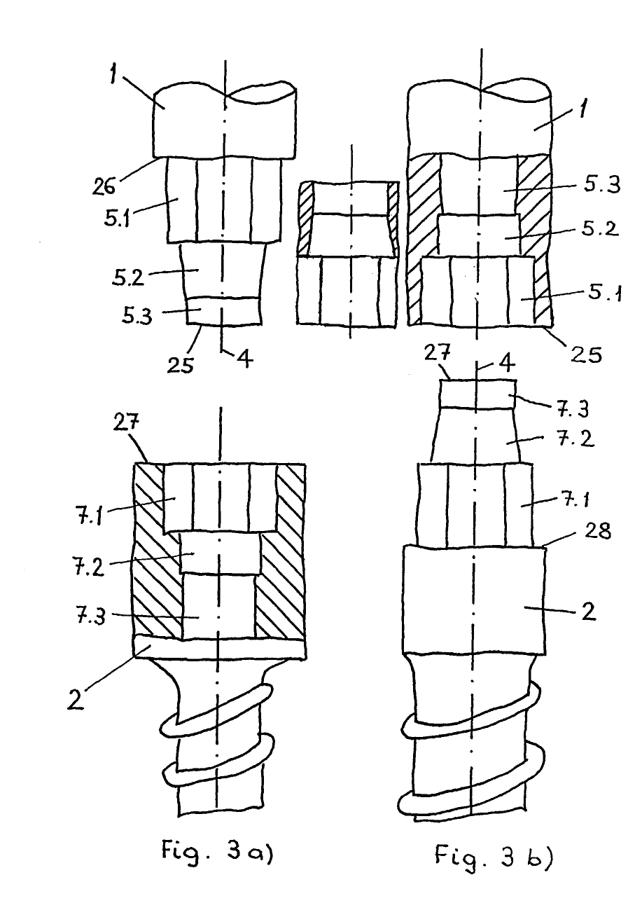

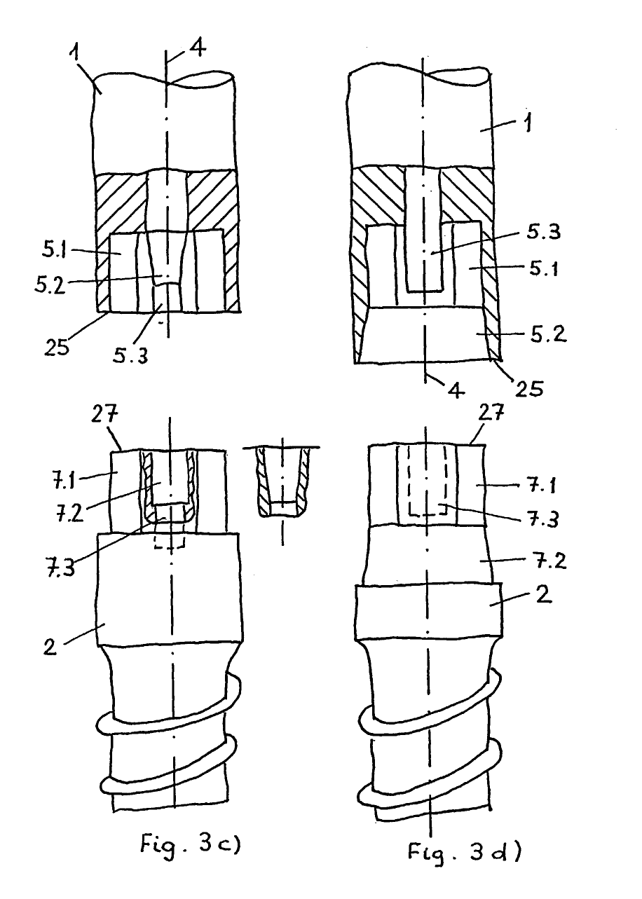

FIGS. 3a) to d) are views showing local sections of different embodiments of the plug-type connection according to the invention;

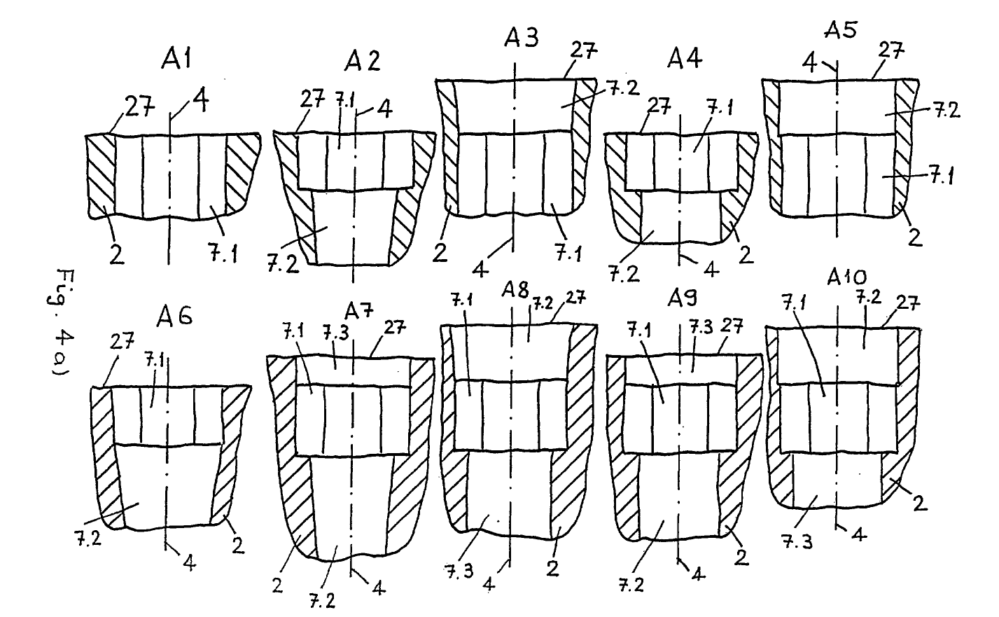

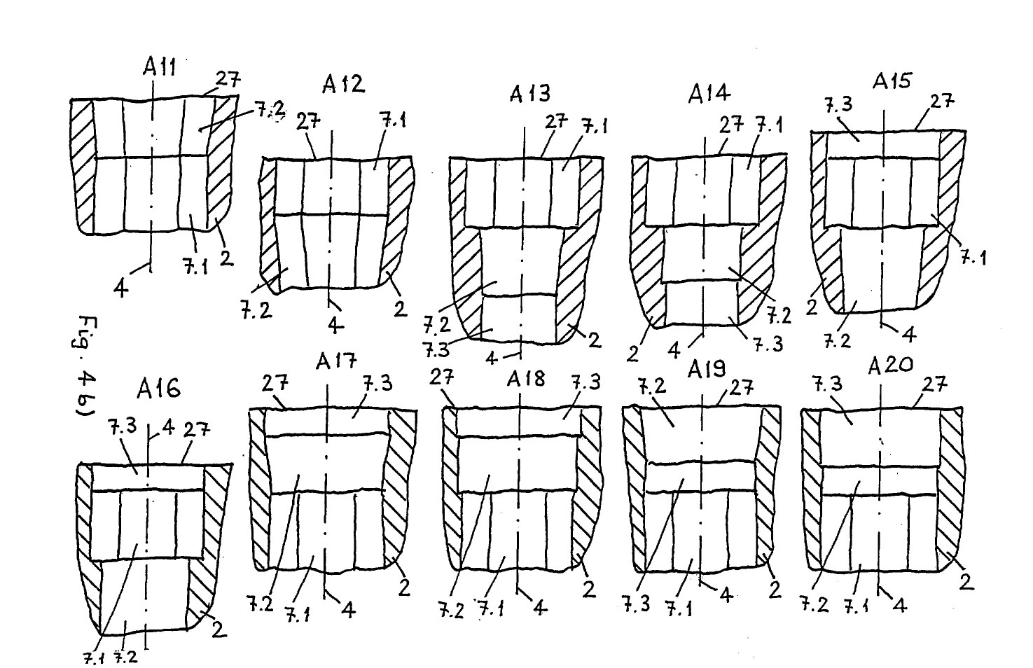

FIGS. 4a) and b) are sectional views of connection zones which contain only recesses as connection segments, according to different embodiments of the inventive, plug-type connection;

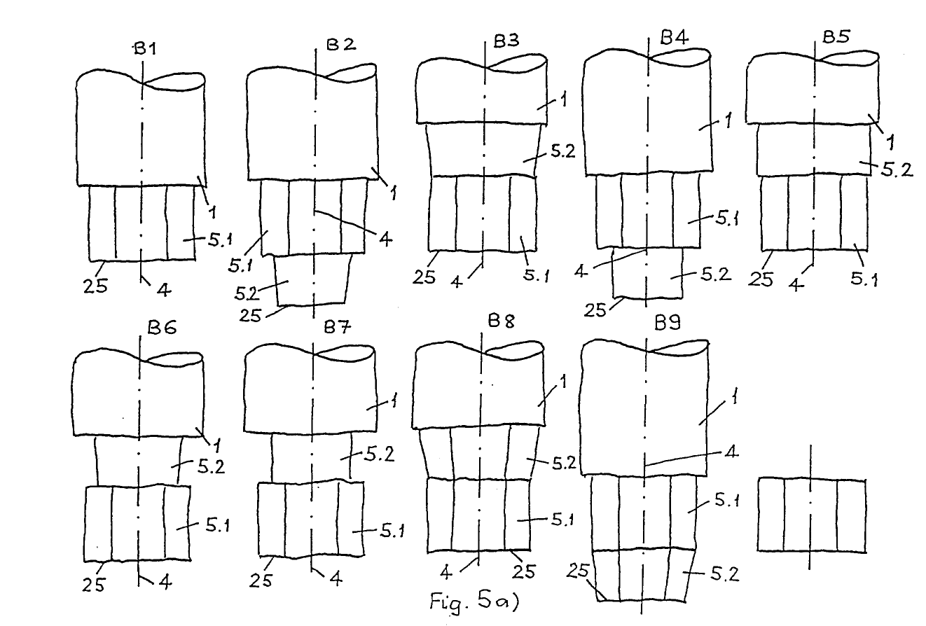

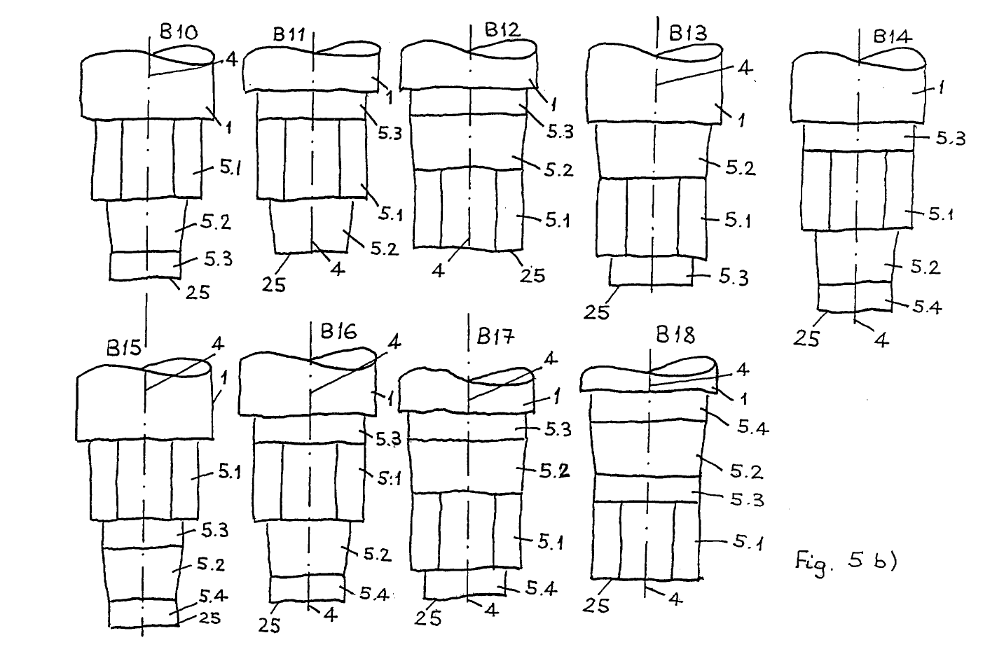

FIGS. 5a) and b) are sectional views of connection zones which contain only tenon-like segments as connection segments, according to different embodiments of the inventive, plug-type connection;

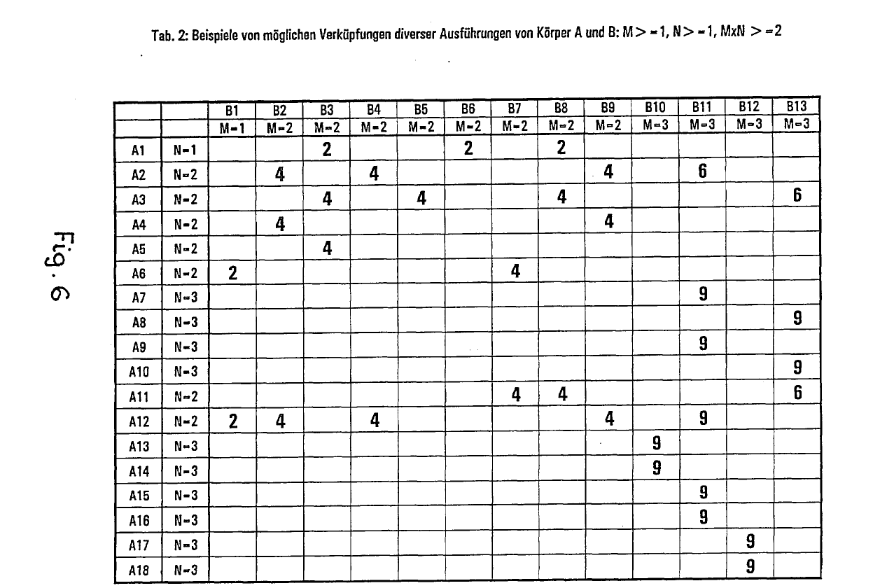

FIG. 6 shows a table representing the possibilities of combining the connection zones according to FIGS. 4a) and b) with the connection zones according to FIGS. 5a) and b);

FIG. 7 shows perspective views of other connection zones according to different embodiments of the inventive plug-type connection in which connection segments containing recesses are combined with connection segments containing tenon-like segments;

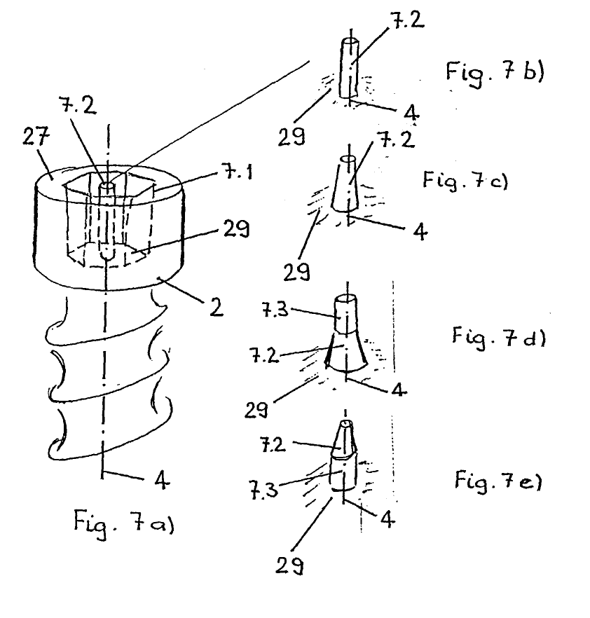

FIG. 8 is a perspective view of one embodiment of the device according to the invention; and

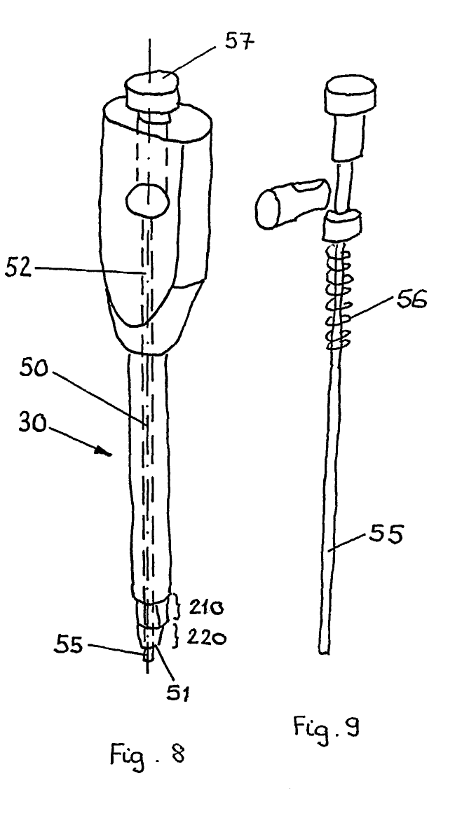

FIG. 9 is a perspective view of the pin serving for pushing away the screw according to the embodiment of the inventive device represented in FIG. 8.

FIGS. 1 and 2 represent an embodiment of the plug-type connection according to the invention which comprises a first body 1 having a first connection zone 3 extending concentrically to a central axis 4 and a second body 2 having a connection zone 6 which equally extends concentrically to said central axis 4. Adjoining its front end 25, the body 1 comprises the first connection zone 3 including three (M=3) connection segments 5.1; 5.2; 5.3, which axially adjoin one another, beginning from the front end 25, and extend over a length L₁. The connection zone 3 is shaped in the form of a shaft segment 21 extending concentrically to the central axis 4. The first connection segment 5.1 with a non-circular cross section Q_{5.1} is shaped in the form of a hexagonal segment 22 and has a width across corners E and a width across flats SW, whereas the first connection segment 5.2 designed for establishing an axially non-positive connection is shaped in the form of a frusto-conical shaft segment 23 having a cone angle of between 2 and 10 degrees, preferably between 3 and 5 degrees. The frusto-conical shaft segment 23 adjoins the front end 25 and, in this portion, has a diameter dₘᵢₙ while in the portion adjoining the hexagonal segment 22 it has a diameter dₘₐₓ. The third connection segment 5.3, shaped in the form of a circular cylindrical shaft segment 24, serves for stabilising the plug-type connection and has a diameter d equal to or greater than the width across flats SW.

The width across flats SW of the connection segment 5.1 with a non-circular cross section Q_{5.1} corresponds to the diameter of the inscribed circle of this cross-section, whereas the width across corners E corresponds to the diameter of the circumscribed circle of the respective cross-section.

FIG. 2 shows the body 2 having a second connection zone 6 which corresponds to the first connection zone 3. Adjoining its top end 27, the body 2 comprises the second connection zone 6 equally including three (N=3) connection segments 7.1; 7.2; 7.3, which axially adjoin one another, beginning from the top end 27, and extend over a length L₂. The connection zone 6 is shaped in the form of a shaft segment 11 extending coaxially to the central axis 4. The second connection segment 7.1 having a cross section Q_{7.1} which is in positive engagement with the cross section Q_{5.1} is shaped in the form of a hexagon socket 12 and is equally provided with a width across corners E and a width across flats SW. The second connection segment 7.3 adjoining the second connection segment 7.1 on the side of the top end 27 is shaped in the form of a circular cylindrical bore segment 14, the diameter of which is D14≧E. The second connection segment 7.2 which adjoins the second connection segment 7.1 on the side opposite to the bore segment 14, is equally shaped in the form of a circular cylindrical bore segment 13 with a diameter D13≦SW and dₘᵢₙ≦D13≦dₘₐₓ.

In FIGS. 1 and 2, the first and second connection segments 5.1 and 7.1 form a positive connection relative to rotation about the central axis 4, whereas the axially non-positive connection is realised by means of the first connection segment 5.2 shaped in the form of a frusto-conical shaft segment 23 which becomes wedged in the second connection segment 7.2 shaped in the form of a circular cylindrical bore segment 13. The stabilisation of the plug-type connection is achieved by the connection, accurately in register with respect to their diameters, of the first connection segment 5.3 shaped in the form of a circular cylindrical shaft segment 24 and the second connection segment 7.3 shaped in the form of a circular cylindrical bore segment 14.

FIGS. 3a) to 3d) show further embodiments of the plug-type connection according to the invention and will be described as follows:

FIG. 3a) The body 1 which, in the present case, represents a screwdriver comprises, in the order mentioned below and beginning at the front end 25, three (M=3) connection segments 5.3; 5.2 and 5.1, extending coaxially to the central axis 4 and axially adjoining one another,

the connection segment 5.1 being shaped in the form of a hexagonal shaft segment with a width across corners E and a width across flats SW;

the connection segment 5.2 being shaped in the form of a frusto-conical shaft segment with dₘₐₓ≦SW; and

the connection segment 5.3 being shaped in the form of a circular cylindrical shaft segment with d≧dₘᵢₙ.

The body 2 which, in the present case, represents a screw comprises, in the order mentioned below and beginning at the top end 27, three (N=3) connection segments 7.1; 7.2 and 7.3, extending coaxially to the central axis 4 and axially adjoining one another,

the connection segment 7.1 being shaped in the form of a hexagonal bore segment with a width across corners E and a width across flats SW;

the connection segment 7.2 being shaped in the form of a circular cylindrical bore segment with d_{7.2}≦SW; and

the connection segment 7.3 being shaped in the form of a circular cylindrical bore segment with d_{7.3}≦d_{7.2}.

FIG. 3b) The body 1 which, in the present case, represents a screwdriver comprises, in the order mentioned below and beginning at the front end 25, three (M=3) connection segments 5.1; 5.2 and 5.3, extending coaxially to the central axis 4 and axially adjoining one another,

the connection segment 5.1 being shaped in the form of a hexagonal bore segment with a width across corners E and a width across flats SW;

the connection segment 5.2 being shaped in the form of a circular cylindrical or frusto-conical bore segment with dₘₐₓ≦SW; and

the connection segment 5.3 being shaped in the form of a circular cylindrical bore segment with d≦dₘᵢₙ.

The body 2 which, in the present case, represents a screw comprises, in the order mentioned below and beginning at the top end 27, three (N 32 3) connection segments 7.3; 7.2 and 7.1, extending coaxially to the central axis 4 and axially adjoining one another,

the connection segment 7.1 being shaped in the form of a hexagonal shaft segment with a width across corners E and a width across flats SW;

the connection segment 7.2 being shaped in the form of a frusto-conical shaft segment (dₘᵢₙ; dₘₐₓ) with dₘₐₓ≦SW; and

the connection segment 7.3 being shaped in the form of a circular cylindrical shaft segment with d≦dₘᵢₙ.

FIG. 3c) The body 1 which, in the present case, represents a screwdriver, comprises three (M=3) connection segments 5.1; 5.2 and 5.3, extending coaxially to the central axis 4,

the connection segment 5.1 being shaped in the form of a hexagonal bore segment with a width across corners E and a width across flats SW;

the connection segment 5.2 being shaped in the form of a frusto-conical shaft segment, adjoining the connection segment, with dₘₐₓ<SW; and

the connection segment 5.3 being shaped in the form of a circular cylindrical shaft segment, adjoining the front end 25, with d≦dₘᵢₙ and the connection segments 5.2 and 5.3 being attached to the body 1 as fixed or axially displaceable shaft segments.

The body 2 which, in the present case, represents a screw, comprises three (N =3) connection segments 7.1; 7.2 and 7.3, extending coaxially to the central axis 4,

the connection segment 7.1 being shaped in the form of a hexagonal shaft segment with a width across corners E and a width across flats SW;

the connection segment 7.2 being shaped in the form of a circular cylindrical bore segment, adjoining the top end 27, with d_{7.2}<SW; and

the connection segment 7.3 being shaped in the form of a circular cylindrical bore segment, adjoining the connection segment 7.2, with d_{7.3}<d_{7.2}.

FIG. 3d) The body 1 which, in the present case, represents a screwdriver, comprises three (M=3) connection segments 5.1; 5.2 and 5.3, extending coaxially to the central axis 4,

the connection segment 5.1 being shaped in the form of a hexagonal bore segment, adjoining the connection segment 5.2, with a width across corners E and a width across flats SW;

the connection segment 5.2 being shaped in the form of a frusto-conical bore segment, adjoining the top end 25, with dₘᵢₙ≧E; and

the connection segment 5.3 being shaped in the form of a circular cylindrical shaft segment with d≦SW and being attached to the body 1 as a fixed or axially displaceable shaft segment.

The body 2 which, in the present case, represents a screw, comprises three (N=3) connection segments 7.1; 7.2 and 7.3, extending coaxially to the central axis 4,

the connection segment 7.1 being shaped in the form of a hexagonal shaft segment, adjoining the top end 27, with a width across corners E and a width across flats SW;

the connection segment 7.2 being shaped in the form of a frusto-conical shaft segment, adjoining the connection segment 7.1, with dₘᵢₙ≧SW; and

the connection segment 7.3 being shaped in the form of a circular cylindrical shaft segment, adjoining the top end 27, with d_{7.3}<SW.

FIGS. 4a), 4b), 5a), and 5b) comprise further embodiments of the plug-type connection according to the invention, realised as bore segments A1 to A20 formed in the body 2 and as shaft segments B1 to B18 formed in the body 1, the respective connection segments being shaped in different forms, as described in the following:

A1: connection segment 7.1 shaped in the form of a hexagon socket;

A2: connection segment 7.1 shaped in the form of a hexagon socket adjoining the top end 27, and connection segment 7.2 shaped in the form of a frusto-conical bore segment, adjoining the connection segment 7.1, with dₘₐₓ≦SW;

A3: connection segment 7.2 shaped in the form of a frusto-conical bore segment, adjoining the top end 27, with dₘᵢₙ≧E, and connection segment 7.1 shaped in the form of a hexagon socket adjoining the connection segment 7.2;

A4: connection segment 7.1 shaped in the form of a hexagon socket adjoining the top end 27, and connection segment 7.2 shaped in the form of a circular cylindrical bore segment, adjoining the connection segment 7.1, with d≦SW;

A5: connection segment 7.2 shaped in the form of a circular cylindrical bore segment, adjoining the top end 27, with d≧E, and connection segment 7.1 shaped in the form of a hexagon socket adjoining the connection segment 7.2;

A6: connection segment 7.1 shaped in the form of a hexagon socket adjoining the top end 27, and connection segment 7.2 shaped in the form of a frusto-conical bore segment, adjoining the connection segment 7.1, with d_{max≦E;}

A7: connection segment 7.3 shaped in the form of a circular cylindrical bore segment, adjoining the top end 27, with a diameter D; connection segment 7.1 shaped in the form of a hexagon socket, adjoining the connection segment 7.3, with E≦D; and connection segment 7.2 shaped in the form of a frusto-conical bore segment, adjoining the connection segment 7.1, with dₘₐₓ≦SW;

A8: connection segment 7.2 shaped in the form of a frusto-conical bore segment, adjoining the top end 27, with dₘᵢₙ≧E; connection segment 7.1 shaped in the form of a hexagon socket, adjoining the connection segment 7.2; and connection segment 7.3 shaped in the form of a circular cylindrical bore segment, adjoining the connection segment 7.1, with d≧SW;

A9: connection segment 7.3 shaped in the form of a circular cylindrical bore segment, adjoining the top end 27, with a diameter D; connection segment 7.1 shaped in the form of a hexagon socket, adjoining the connection segment 7.3, with E≦D; and connection segment 7.2 shaped in the form of a circular cylindrical bore segment, adjoining the connection segment 7.1, with d≦SW;

A10: connection segment 7.3 shaped in the form of a circular cylindrical bore segment, adjoining the top end 27, with a diameter D; connection segment 7.1 shaped in the form of a hexagon socket, adjoining the connection segment 7.3, with E<D; and connection segment 7.2 shaped in the form of a circular cylindrical bore segment, adjoining the connection segment 7.1, with d≦SW;

A11: connection segment 7.2 shaped in the form of a conical hexagon socket adjoining the top end 27, and connection segment 7.1 shaped in the form of a prismatic hexagon socket and adjoined to the connection segment 7.2 by means of an aligning transition;

A12: connection segment 7.1 shaped in the form of a prismatic hexagon socket adjoining the top end 27, and connection segment 7.2 shaped in the form of a conical hexagon socket adjoined to the connection segment 7.1 by means of an aligning transition;

A13: connection segment 7.1 shaped in the form of a hexagon socket adjoining the top end 27; connection segment 7.2 shaped in the form of a frusto-conical bore segment, adjoining the connection segment 7.1, with dₘₐₓ≦SW; and connection segment 7.3 shaped in the form of a circular cylindrical bore segment, adjoining the connection segment 7.2, with d≦dₘₐₓ;

A14: connection segment 7.1 shaped in the form of a hexagon socket adjoining the top end 27; connection segment 7.2 shaped in the form of a circular cylindrical bore segment, adjoining the connection segment 7.1, with D≦SW; and connection segment 7.3 shaped in the form of a circular cylindrical bore segment, adjoining the connection segment 7.2, with d≦D;

A15: identical with A7;

A16: connection segment 7.3 shaped in the form of a circular cylindrical bore segment, adjoining the top end 27, with D≧E; connection segment 7.1 shaped in the form of a hexagon socket, adjoining the connection segment 7.3; and connection segment 7.2 shaped in the form of a circular cylindrical bore segment, adjoining the connection segment 7.1;

A17: connection segment 7.3 shaped in the form of a circular cylindrical bore segment, adjoining the top end 27, with D≧dₘₐₓ; connection segment 7.2 shaped in the form of a frusto-conical bore segment, adjoining the connection segment 7.3; and connection segment 7.1 shaped in the form of a hexagon socket, adjoining the connection segment 7.2, with E≦dₘᵢₙ;

A18: connection segment 7.3 shaped in the form of a circular cylindrical bore segment, adjoining the top end 27, with D>d; connection segment 7.2 shaped in the form of a circular cylindrical bore segment, adjoining the connection segment 7.3, with d<D; and connection segment 7.1 shaped in the form of a hexagon socket, adjoining the connection segment 7.2, with E<d;

A19: connection segment 7.2 shaped in the form of a frusto-conical bore segment, adjoining the top end 27; connection segment 7.3 shaped in the form of a circular cylindrical bore segment, adjoining the connection segment 7.2, with d≦dₘᵢₙ; and connection segment 7.1 shaped in the form of a hexagon socket, adjoining the connection segment 7.3, with E≦d;

A20: connection segment 7.3 shaped in the form of a circular cylindrical bore segment, adjoining the top end 27, with a diameter D; connection segment 7.2 shaped in the form of a circular cylindrical bore segment, adjoining the connection segment 7.3, with d≦D; and connection segment 7.1 shaped in the form of a hexagon socket, adjoining the connection segment 7.3, with E≦d.

In FIG. 5a) the elements B1 to B5 differ from the elements A1 to A5 illustrated in FIG. 4a) in so far as the respective bore segments are replaced by corresponding shaft segments, the remaining connection segments being shaped in different forms, as described in the following:

B6: connection segment 5.1 shaped in the form of a hexagonal segment adjoining the front end 25, and connection segment 5.2 shaped in the form of a frusto-conical shaft segment, adjoining the connection segment 5.1, with dₘₐₓ>SW;

B7: connection segment 5.1 shaped in the form of a hexagonal segment adjoining the front end 25, and connection segment 5.2 shaped in the form of a circular cylindrical shaft segment, adjoining the connection segment 5.1, with d≧SW;

B8: differs from the element A11 only in so far as the bore segments are replaced by corresponding shaft segments;

B9: differs from the element A12 only in so far as the bore segments are replaced by corresponding shaft segments;

B10: connection segment 5.3 shaped in the form of a circular cylindrical shaft segment, adjoining the front end 25, with a diameter d; connection segment 5.2 shaped in the form of a frusto-conical shaft segment, adjoining the connection segment 5.3, with dₘᵢₙ≧d; and connection segment 5.1 shaped in the form of a hexagonal segment, adjoining the connection segment 5.2, with SW≧dₘᵢₙ;

B11: differs from the element A7 only in so far as the bore segments are replaced by corresponding shaft segments;

B12: differs from the element A17 only in so far as the bore segments are replaced by corresponding shaft segments;

B13: differs from the element A8 only in so far as the bore segments are replaced by corresponding shaft segments;

B14: differs from the element B11 only in so far as, adjoining the front end 25, a connection segment 5.4 shaped in the form of a circular cylindrical shaft segment with d≦dₘᵢₙ has been added to the connection segment 5.2;

B15: connection segment 5.4 shaped in the form of a circular cylindrical shaft segment, adjoining the front end 25, with a diameter d; connection segment 5.2 shaped in the form of a frusto-conical shaft segment, adjoining the connection segment 5.4, with dₘᵢₙ≧d; connection segment 5.3 shaped in the form of a circular cylindrical shaft segment, adjoining the connection segment 5.2, with D≧dₘₐₓ; and connection segment 5.1 shaped in the form of a hexagonal segment, adjoining the connection segment 5.3, with SW≧D;

B16: is identical with B14;

B17: differs from the element B12 only in so far as, adjoining the front end 25, a connection segment 5.4 shaped in the form of a circular cylindrical shaft segment with d≦SW has been added to the connection segment 5.1;

B18: differs from the element B12 only in so far as a connection segment 5.3 shaped in the form of a circular cylindrical shaft segment with a diameter D meeting the condition E≦D≦dₘᵢₙ has been inserted between the connection segments 5.1 and 5.2.

FIG. 6 is a tabular representation of the combinations of the first and second connection segments shaped in the forms of shaft segments B1 to B18 and bore segments A1 to A20, as represented in FIGS. 4a), 4b), 5a), and 5b).

FIGS. 7a) to 7e) show further embodiments of the device according to the invention. These are exemplified by means of a body 2 which is realised in the form of a screw. A connection segment 7.1 shaped in the form of a hexagonal bore segment adjoins the top end 27 and reaches down to the bottom 29. On the bottom 29, one or two connection segments shaped in the form of shaft segments are arranged which extend towards the top end 27. In FIG. 7a) and 7b), the connection segment 7.2 is shaped in the form of a circular cylindrical shaft segment; in FIG. 7c), the connection segment 7.2 is shaped in the form of a frusto-conical shaft segment; in FIG. 7.d) the connection segment 7.2 is shaped in the form of a frusto-conical shaft segment extending upright from the bottom 29 and adjoined by a connection segment 7.3 shaped in the form of a circular cylindrical shaft segment (d≦dₘᵢₙ); and in FIG. 7e) the connection segment 7.3 is shaped in the form of a circular cylindrical shaft segment extending upright from the bottom 29 and adjoined by a connection segment 7.2 shaped in the form of a frusto-conical shaft segment (dₘₐₓ≦d).

FIGS. 8 and 9 show an embodiment of the device according to the invention in which the body 1 is shaped in the form of a screwdriver 30 which serves for screwing in or screwing out a body 2 (FIG. 2) shaped in the form of a screw. The screwdriver 30 comprises a longitudinal axis 50, a front end 51 directed towards the body 2 (FIG. 2), and a bore 52 which extends coaxially to the longitudinal axis 50 and is open towards the front end 51. An axially displaceable pin 55 is positioned within the bore 52 which is pushed within the bore 52 in the direction of the front end 51 by means of a spring 56. The pin 55 is pushed towards the front end 51 until it abuts against the bottom of the bore segment 11 of the body 2 (FIG. 2). Furthermore, by pushing on its rear end 57, the pin 55 may be displaced at least sufficiently far in the direction of the front end 51 so that the first and second connection segments 5.1 and 7.1, which are in positive engagement with each other and shaped respectively as a hexagon insert bit 210 formed in the screwdriver and as a hexagon socket 12 formed in the screw (FIG. 2), cease to be in mutual engagement and, consequently, also the first connection segment 5.2 (FIG. 1) of the screwdriver 30, shaped in the form of a frusto-conical shaft segment 220, becomes completely detached from the bore segment 11 of the body 2 shaped in the form of a screw.

1. A plug-type connection for releasably connecting two bodies (1) and (2),
A) the first body (1) comprising a first connection zone (3) having a central axis (4), a length (L₁), and M (M>1) first connection segment (5.1, . . . , 5.m) arranged coaxially to the central axis (4);
B) the second body (2) comprising a second connection zone (6) having a central axis (4), a length (L₂), and N (N>1) second connection segment (7.1, . . . , 7.m) arranged coaxially to the central axis (4), so that
C) the first connection zone (3) and the second connection zone (6) form a plug-type connection extending coaxially to the central axis (4); and
D) the first connection zone (3) has at least one first connection segment (5.1) with a non-circular cross-section Q_{5.1} and the second connection zone (6) has at least one second connection segment (7.1) with a cross-section Q_{7.1} in positive engagement with said cross-section Q_{5.1}, whereby a rotationally positive engagement relative to the central axis (4) is achievable,
characterised in that
E) M×N is ≧2; and
F) at least one first connection segment (5.1, . . . , 5.m) and at least one second connection segment (7.1, . . . , 7.n) form an axially non-positive engagement relative to the central axis (4):

2. A plug-type connection as claimed in claim 1, characterised in that the non-positive engagement is formed by at least one connection segment (5.1, . . . , 5.m; 7.1, . . . , 7.n) having an axially tapering cross-section Q.

3. A plug-type connection as claimed in claim 1 or 2, characterised in that all connection segments (7.1, . . . , 7n) axially adjoin one another with respect to the central axis (4) and differ from one another in shape.

4. A plug-type connection as claimed in claim 1 or 3, characterised in that all connection segments (5.1, . . . , 5.m) axially adjoin one another with respect to the central axis (4) and differ from one another in shape.

5. A plug-type connection as claimed in claim 1, characterised in that the connection segments (5.1, . . . , 5.m) consist of a combination of shaft segments and bore segments.

6. A plug-type connection as claimed in any of the claims 1 to 5, characterised in that the first connection zone (3) consists of a single piece.

7. A plug-type connection as claimed in any of the claims 1 to 5, characterised in that the first connection zone (3) consists of several pieces, preferably of two pieces.

8. A plug-type connection as claimed in claim 7, characterised in that at least one first connection segment (5.i, 1≦i≦m) is coaxially displaceable relative to the other first connection segments (5.j, j≠i).

9. A plug-type connection as claimed in claim 4, characterised in that the second connection segments (7.1, . . . , 7.n) consist of a combination of shaft segments and bore segments.

10. A plug-type connection as claimed in any of the claims 1 to 9, characterised in that the second connection zone (6) consists of a single piece.

11. A plug-type connection as claimed in any of the claims 1 to 9, characterised in that the second connection zone (6) consists of several pieces, preferably of two pieces.

12. A plug-type connection as claimed in claim 11, characterised in that at least one second connection segment (7.i, 1≦i≦n) is coaxially displaceable relative to the other second connection segments (7.j, j≠i).

13. A plug-type connection as claimed in any of the claims 1 to 12, characterised in that at least one of the first connection segments (5.1, . . . , 5.i) forming a rotationally positive engagement with respect to the central axis (4) at the same time forms a non-positive engagement with one of the second connection segments (7.1, . . . , 7.n) which is effective parallel to the central axis (4).

14. A plug-type connection as claimed in any of the claims 1 to 13, characterised in that at least one of the second connection segments (7.1, . . . , 7.j) forming a rotationally positive engagement relative to the central axis (4) at the same time forms a non-positive engagement with one of the second connection segments (5.1, . . . , 5.m) which is effective parallel to the central axis (4).

15. A plug-type connection as claimed in any of the claims 1 to 14, characterised in that the first and second connection segments (5.1, . . . , 5.i; 7.1, . . . , 7.j) forming the rotationally positive engagement relative to the central axis (4) are provided with complementarily polygonal cross-sections (8.1, . . . , 8.i; 9.1, . . . , 9.j).

16. A plug-type connection as claimed in claim 15, characterised in that the cross-sections (8.1, . . . , 8.i; 9.1, . . . , 9.j) are hexagonal.

17. A plug-type connection as claimed in any of the claims 1 to 14, characterised in that the first and second connection segments (5.1, . . . , 5.i; 7.1, . . . , 7.j) forming the rotationally positive engagement relative to the central axis (4) are provided with complementarily star-shaped cross-sections (8.1, . . . , 8.i; 9.1, . . . , 9.j) (star-shaped socket with six branches).

18. A plug-type connection as claimed in any of the claims 1 to 14, characterised in that the first and second connection segments (5.1, . . . , 5.i; 7.1, . . . , 7.j) forming the rotationally positive engagement relative to the central axis (4) are provided with complementarily elliptical cross-sections (8.1, . . . , 8.i; 9.1, . . . , 9.j).

19. A plug-type connection as claimed in any of the claims 1 to 18, characterised in that M≧2 and N≧2.

20. A plug-type connection as claimed in any of the claims 1 to 18, characterised in that N=M+1 and M≧1.

21. A plug-type connection as claimed in any of the claims 1 to 18, characterised in that M=N+1 and N>1.

22. A plug-type connection as claimed in claim 20, characterised in that M=1 and N=2.

23. A plug-type connection as claimed in claim 21, characterised in that M=2 and N=1.

24. A plug-type connection as claimed in claim 19, characterised in that M=N.

25. A plug-type connection as claimed in claim 24, characterised in that M=2.

26. A plug-type connection as claimed in claim 24, characterised in that M=3.

27. A plug-type connection as claimed in claim 25, characterised in that the two second connection segments (7.1; 7.2) of the second body (2) comprise a bore segment (11) including a hexagon socket (12) having a width across flats SW, and a cylindrical bore segment (13) having a diameter d<SW.

28. A plug-type connection as claimed in claim 27, characterised in that the two first connection segments (5.1; 5.2) of the body (1) comprise a shaft segment (21) including a hexagonal segment (22) and a frusto-conical shaft segment (23) having a cone angle α of between 2 and 10 degrees, the greatest diameter Dₘₐₓ of the truncated cone being greater, and the smallest diameter Dₘᵢₙ being smaller than the diameter d of the cylindrical bore segment (13).

29. A plug-type connection as claimed in claim 26, characterised in that the three second connection segments (7.1; 7.2; 7.3) of the body (2) comprise a bore segment (11) including a circular cylindrical bore segment (14) having a diameter D₁₄, a hexagon socket (12) having a width across flats SW and a width across corners E, and a cylindrical bore segment (13) having a diameter d≦SW, the diameter D₁₄ being ≧E.

30. A plug-type connection as claimed in claim 29, characterised in that the three first connection segments (5.1; 5.2; 5.3) of the body (1) comprise a shaft segment (21) including a circular cylindrical shaft segment (24) having a diameter D₂₄, a hexagonal segment (22) having a width across corners E and a width across flats SW, and a frusto-conical shaft segment (23) having a cone angle α of between 2 and 10 degrees, the diameter D₂₄ being=D₁₄ and the greatest diameter Dₘₐₓ of the truncated cone being greater, and the smallest diameter Dₘᵢₙ being smaller than the diameter d of the cylindrical bore segment (13).

31. A plug-type connection as claimed in claim 28 or 30, characterised in that the cone angle α is between 3 and 5 degrees.

32. A plug-type connection as claimed in claim 19, characterised in that the two second connection segments (7.1; 7.2) of the body (2) comprise a bore segment (14) including a star-shaped socket (15) with six branches having an inside diameter di, and a cylindrical bore segment (13) having a diameter d≧dᵢ.

33. A plug-type connection as claimed in claim 19, characterised in that the two connection segments (5.1; 5.2) of the body (1) comprise a shaft segment (24) including a star-shaped segment (25) with six branches and a frusto-conical shaft segment (23) having a cone angle α of between 2 and 10 degrees, the greatest diameter Dₘₐₓ of the truncated cone being greater, and the smallest diameter Dₘᵢₙ being smaller than the diameter d of the cylindrical bore segment (13).

34. A plug-type connection as claimed in claim 26, characterised in that the three second connection segments (7.1; 7.2; 7.3) of the body (2) comprise a bore segment (11) including a circular cylindrical bore segment (14) having a diameter D₁₄, a star-shaped socket with six branches having an inside diameter dᵢ and an outside diameter dₐ, and a cylindrical bore segment (13) having a diameter d≦dᵢ, the diameter D being ≧dₐ.

35. A plug-type connection as claimed in claim 34, characterised in that the three first connection segments (5.1; 5.2; 5.3) of the body (1) comprise a shaft segment (21) including a circular cylindrical shaft segment (24) having a diameter D₂₄, a star-shaped segment with six branches having an inside diameter dᵢ and an outside diameter dₐ, and a frusto-conical shaft segment (23) having a cone angle α of between 2 and 10 degrees, the diameter D₂₄ being =D₁₄ and the greatest diameter Dₘₐₓ of the truncated cone being greater, and the smallest diameter Dₘᵢₙ being smaller than the diameter d of the cylindrical bore segment (13).

36. A plug-type connection as claimed in claim 33 or 35, characterised in that the cone angle α is between 3 and 5 degrees.

37. A plug-type connection as claimed in any of the claims 1 to 25, characterised in that, in cases in which N≧2, at least one of the connection segments (7.1, . . . , 7.n) of the body (2) has a conically tapered form converging in the direction in which the plugging takes place.

38. A plug-type connection as claimed in any of the claims 1 to 37, characterised in that, in cases in which M≧2, at least one of the connection segments (5.1, . . . , 5.m) of the body (1) has a conically tapered form converging in the direction in which the plugging takes place.

39. A plug-type connection as claimed in any of the claims 1 to 38, characterised in that, in cases in which N≧3, at least one of the connection segments (7.1, . . . , 7.n) has a cylindrical form.

40. A plug-type connection as claimed in any of the claims 1 to 39, characterised in that, in cases in which M≧3, at least one of the connection segments (5.1, . . . , 5.m) of the frontal part of the body (1) has a cylindrical form.

41. A device including a plug-type connection as claimed in any of the claims 1 to 40, characterised in that the body (1) is a screwdriver (30) and the body (2) a screw (40).

42. A device including a plug-type connection as claimed in any of the claims 1 to 40, characterised in that the body (2) is a screwdriver (30) and the body (1) a screw (40).

43. A device as claimed in claim 41, characterised in that the body (1) comprises a longitudinal axis (50), a front end (51) facing the body (2), and a bore (52) which is open towards the front end (51) and extends coaxially to the longitudinal axis (50).

44. A device as claimed in claim 42, characterised in that the body (2) comprises a longitudinal axis (60), a front end (61) facing the body (1), and a bore (52) which is open towards the front end (61) and extends coaxially to the longitudinal axis (60).

45. A device as claimed in claim 43 or 44, characterised in that an axially displaceable pin (55) is positioned in the bore (52).

46. A device as claimed in claim 45, characterised in that the pin (55) is pushed into the bore (52) by means of a spring (56).

47. A device as claimed in claim 45 or 46, characterised in that the pin (55) may be at least pushed sufficiently far in the direction of the front end (51; 61) so as to abut against the bottom of the bore segment (11) formed in the body (2).

48. A device as claimed in claim 47, characterized in that the pin (55) may be at least pushed sufficiently far in the direction of the front end (51; 61) so that the segments (12, 210) of the bore segment (11) formed in body (2), on the one hand, and of the frontal part of body (1), on the other, which form a positive engagement, cease to be in mutual engagement.

49. A device as claimed in claim 46, characterised in that the pin (55) may be at least pushed forward sufficiently far so as to allow the frontal part 210 of the body (1) to be entirely pushed out of the bore segment (11) formed in the body (2).
